(19) United States
(12) Patent Application Publication
(10) Pub. No.: US 2016/0175606 A1
(43) Pub. Date: Jun. 23, 2016

(54) OPTICAL STIMULATOR USING ELECTROCHROMISM

(71) Applicant: GWANGJU INSTITUTE OF SCIENCE AND TECHNOLOGY, Gwangju (KR)

(72) Inventors: Sung-Joon CHO, Gwangju (KR); Dong-Hak BYUN, Gwangju (KR); So-Hee KIM, Gwangju (KR); Keong-Hwan OH, Gwangju (KR)

(21) Appl. No.: 14/942,945

(22) Filed: Nov. 16, 2015

(30) Foreign Application Priority Data
Dec. 17, 2014 (KR) 10-2014-0182399

(51) Int. Cl. A61N 5/06

(52) U.S. Cl. CPC A61N 5/0601; A61N 5/0622; A61N 2005/0612; A61N 2005/063

(57) ABSTRACT

Disclosed herein is an optical stimulator inserted into a biological tissue for obtaining a neural signal by stimulating a neuron with a propagating light. The optical stimulator includes: a main unit comprising electrodes, and electrical terminals connected to the electrodes; a waveguide extended from a side of the main unit and comprising electrochromic films; and a light source disposed in the main unit and producing light along a direction in which the electrochromic films formed in the waveguide are arranged. Each of the electrical terminals is connected to the respective electrochromic films, and the electrochromic films change a propagation path of light reaching the electrochromic films upon a voltage being applied thereto from the electrical terminals, to stimulate a neuron.

### CROSS-REFERENCE TO RELATED APPLICATIONS

This application claims the priority of Korean Patent Application No. KR 10-2014-0182399 filed on Dec. 17, 2014, in the Korean Intellectual Property Office, the disclosure of which is incorporated herein by reference.

### BACKGROUND

1. Technical Field

The present disclosure relates to an optical stimulator capable of changing an optical path to a target neuron using electrochromism, especially using electrochromic films.

2. Description of the Related Art

Optogenetics is a biological technique that is attracting neuroscientists or engineer's attention as it exhibits a novel stimulation method that overcomes shortcomings of the electrical simulation method in the related art. In the existing electrical stimulation method, it is difficult to selectively stimulate a focused area of interest in neural tissues. In contrast, the neural stimulation method using optogenetics modifies the activities of neurons that are genetically engineered such that their ion channels respond selectively to light of particular wavelengths.

When a genetically-modified neuron is exposed to light of a particular wavelength, it reacts with the light and opens or closes its ion channels resulting in increase or decrease in action potential generation. Accordingly, it is possible to stimulate a single neuron, and thus more precisely stimulate a neuron than the existing electrical stimulation method.

As a stimulation device used in the optogenetics, an optical fiber, an LED, an OLED, etc., are used. OLEDs and LEDs are advantageous in that they only require a power source and thus have small size. However, the light emitted from an OLED or an LED reaches a relatively large area, compared to that emitted from an optical fiber. In addition, it is highly costly to fabricate micro and nano-sized LED or OLED. Moreover, the light emitted from an LED and an OLED has a lower intensity than the light transmitted via an optical fiber, and thus may fail to reach a threshold value at which a genetically-modified neuron reacts.

A stimulation device using an optical fiber includes an optical fiber inserted into a subject and connected to an external light source such as a laser device. The light source is connected to the optical fiber only when stimulation is carried out. Stimulation can be carried out only by the tip of the optical fiber inserted into a brain or a neuron. Accordingly, it is difficult to use the device at another position or area when the device is inserted into a brain for a chronic experiment. Changing of positions is only possible in an acute experiment.

Further, if the light source is installed closely to a target position, heat generated in its use may adversely affect neurons and may increase thermal noises while recording neuronal signal. Thus, the application range of optogenetics may be limited with existing devices having an optical fiber.

### SUMMARY

An aspect of the present disclosure is to provide an optical stimulator capable of performing stimulation of neurons at several positions in the depth direction rather than a single site, for both a chronic experiment and an acute experiment using optogenetics in the neural system.

Another aspect of the present disclosure is to provide an optical stimulator capable of recording neuronal signals in response to stimulation on neurons more closely to the neurons.

Another aspect of the present disclosure is to provide an optical stimulator capable of adjusting the intensity of light and adjusting light stimulation in several directions even at the same depth by introducing electrochromism technology.

In accordance with one aspect of the present disclosure, an optical stimulator inserted into a biological tissue for obtaining neuronal signals by stimulating a small group of neurons with a propagating light includes: a main unit comprising electrodes, and electrical terminals connected to the electrodes; a waveguide extended from a side of the main unit and comprising electrochromic films; and a light source disposed in the main unit and producing light along a direction in which the electrochromic films formed in the waveguide are arranged. Each of the electrical terminals is connected to the respective electrochromic films, and the electrochromic films change a propagation path of light reaching the electrochromic films upon a voltage being applied thereto from the electrical terminals, to stimulate a neuron.

The electrochromic films may include first electrochromic films for changing an intensity of light, and second electrochromic films for reflecting light reaching it. The first and second electrochromic films may be arranged in the waveguide.

### BRIEF DESCRIPTION OF THE DRAWINGS

The above and other aspects, features and advantages of the present invention will become apparent from the following description of exemplary embodiments given in conjunction with the accompanying drawings, in which:

FIG. 1 is a perspective view of an optical stimulator according to an exemplary embodiment of the present disclosure;

FIG. 2 is an enlarged perspective view of a portion of the optical stimulator according to the exemplary embodiment of the present disclosure;

FIG. 3 is a view for illustrating a principle that the optical stimulator according to the exemplary embodiment of the present disclosure performs optical stimulation;

FIG. 4 is a view for illustrating a principle that the optical stimulator according to the exemplary embodiment of the present disclosure adjusts the intensity of light;

FIG. 5 is a view for illustrating a principle that the optical stimulator according to the exemplary embodiment of the present disclosure changes the path of light; and

FIG. 6 is a view for comparing an existing optical stimulator with the optical stimulator according to the exemplary embodiment of the present disclosure.

### DETAILED DESCRIPTION

Hereinafter, exemplary embodiments of the present disclosure will be described in detail with reference to the accompanying drawings. However, the present disclosure is not limited to these exemplary embodiments. In describing the present disclosure, a description of a well-known function or configuration may be omitted in order not to obscure the gist of the present disclosure.

To overcome limitations of existing optical stimulators used in optogenetics, the present disclosure is directed to an optical stimulator capable of adjusting the intensity of light or selectively changing the path of light at a point of a substrate by introducing the electrochromism technology.

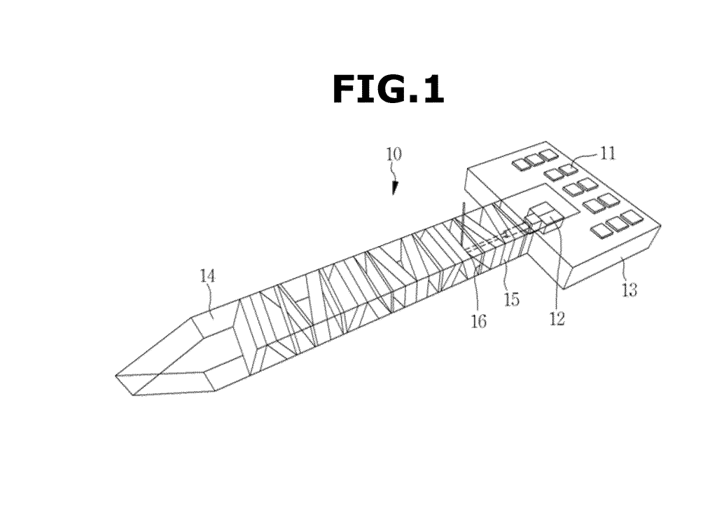

FIG. 1 is a perspective view of an optical stimulator according to an exemplary embodiment of the present disclosure.

Referring to FIG. 1, the optical stimulator 10 according to the exemplary embodiment of the present disclosure may include a main unit 13, electrical terminals 11, a light source 12, a waveguide 14, and electrochromic films 15 and 16.

The main unit 13 has electrodes (not shown) therein and the electrical terminals 11 connected to the electrodes. Each of the electrical terminals 11 includes a conductive line (not shown). The conductive line is extended along the waveguide 14 in which the electrochromic films 15 and 16 are arranged and may be connected to the electrochromic films 15 and 16. Each of the electrical terminals 11 applies a voltage to the respective electrochromic films. The number of the electrical terminals 11 may be equal to the number of the electrochromic films 15 and 16.

The waveguide 14 that guides light produced in the light source 12 may be connected to one side of the main unit 13, with a predetermined length. The waveguide 14 has the electrochromic films 15 and 16 therein to determine a propagation direction of light. The waveguide 14 is inserted into a biological tissue and guides the light passing through it 14 to stimulate a target neuron.

The light source 12 that produces light of a particular wavelength may be disposed at the base of the waveguide 14. The light may be emitted from the source 12 to propagate along the direction in which the waveguide 14 is extended.

In the waveguide 14, a plurality of electrochromic films 15 and 16 may be disposed along the path in which the light produced in the light source 12 propagates such that the films make a predetermined angle, with a face of one of the film in contact with a face of another. The number of the electrochromic films 15 and 16 and the angle at which the films are disposed may vary depending on the type of a target neuron and the location of a stimulation point.

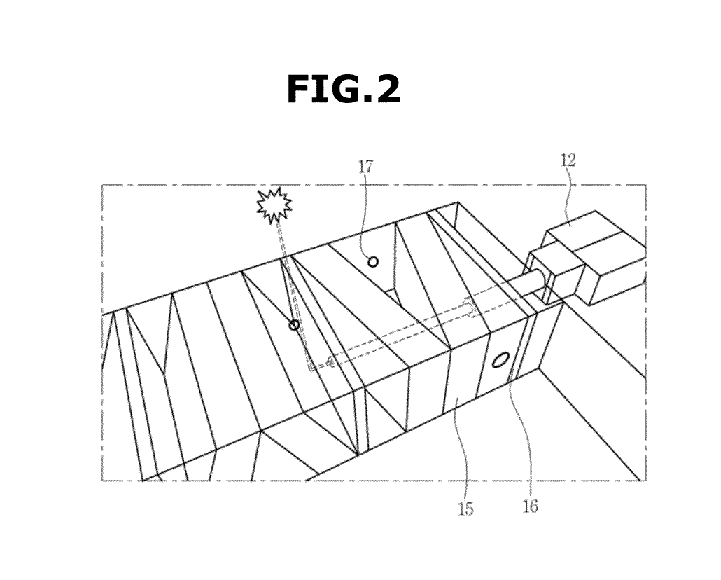

FIG. 2 is a perspective view of a portion of the optical stimulator according to the exemplary embodiment of the present disclosure.

Referring to FIG. 2 in which a portion of the waveguide 14 of the optical stimulator according to the exemplary embodiment of the present disclosure is enlarged, the electrochromic films 15 and 16 may be disposed in the waveguide 14 with an angle with respect to the propagation direction of light.

Specifically, the electrochromic films 15 and 16 may include first electrochromic films 16 for adjusting the intensity of propagating light, and second electrochromic films 15 for changing the path of propagating light.

The first electrochromic films 16 are transparent films whose color is changed depending on a voltage applied thereto, thereby changing the intensity of the propagating light. The first electrochromic films 16 may have a face perpendicular to the propagation direction of light in the waveguide 14. The second electrochromic films 15 are reflective films whose color is changed depending on a voltage applied thereto, thereby changing the path of the propagating light. The second electrochromic films 15 in the waveguide 14 may have a face making an angle with the propagation direction of light, with a face of one in contact with a face of another.

However, this is merely for easy manufacturing, and the films may not have a face in contact with a face of another. Any array of electrochromic films falls within the scope of the present disclosure. The light reflected from the second electrochromic films 15 propagates along the waveguide 14. A recording electrode 17 may be disposed on the waveguide 14 where the light propagates, for measuring a signal produced from a target neuron.

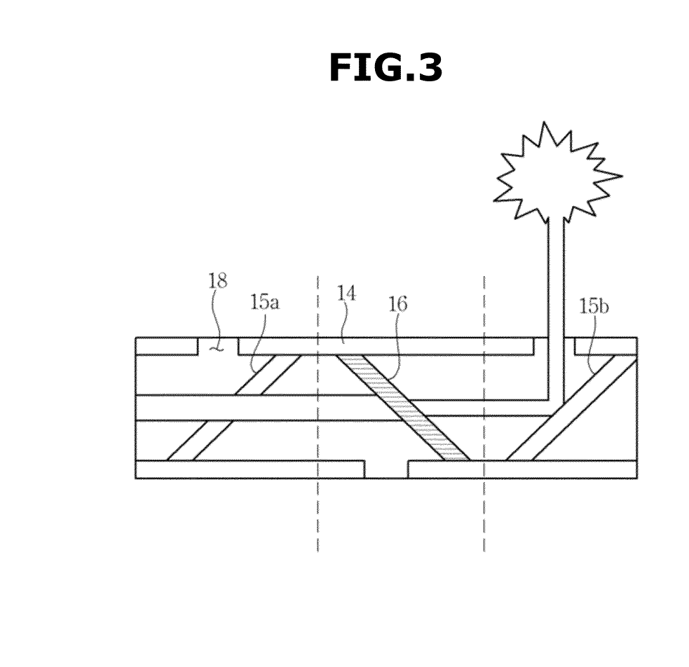

FIG. 3 is a view for illustrating a principle that the optical stimulator according to the exemplary embodiment of the present disclosure performs optical stimulation. FIG. 3 is the view of the inside of the waveguide 14 of the optical stimulator when viewed from its side. In the waveguide 14, the first electrochromic films 16 and the second electrochromic films 15a and 15b are arranged sequentially at a predetermined angle.

Electrochromism is the phenomenon displayed by some materials of reversibly changing color by using bursts of charge to cause electrochemical redox reactions in electrochromic materials. By making use of such phenomenon, the exemplary embodiment of the present disclosure may employ polymer films made of WO₃, TiO₃, IrO₂, NiO, MoO₃, etc., that may become a transparent film, an opaque film, or a reflective film displaying a metallic color, by adjusting voltage applied thereto.

The first electrochromic films 16 may be, but is not limited to, transparent polymer electrochromic films whose color is changed depending on a voltage applied thereto, such as PBEDOTPh.

Table 1 shows color change versus voltage applied.

TABLE 1

| Voltage Applied | | |
|---|---|---|
| (V vs. Ag/Ag⁺) | Oxidation State | Color |
| 1.0 | P(BEDOTPh^{1.7+}-2 V²⁺) | Transparent Pale Blue |
| 0.1 | P(BEDOTPh^{0.6+}-2 V²⁺) | Bisque |
| −0.5 | P(BEDOTPh0-2 V²⁺) | Magenta |
| −0.9 | P(BEDOTPh0-2 V⁺) | Purple |
| −1.4 | P(BEDOTPh0-2 V⁰) | Crimson |

As can be seen from Table 1, the first electrochromic films 16 exhibits different colors depending on the voltage applied thereto. Light is selectively transmitted depending on a changed color. Thus, the intensity of light passing through the first electrochromic films 16 may be adjusted by applying a predetermined potential to the first electrochromic films 16, thereby adjusting the intensity of optical stimulation transmitted to a neuron.

The second electrochromic films 15a and 15b are reflective electrochromic films, and may be produced, but is not limited to, by synthesizing a PET film, an ITO film, liquid crystal molecules, etc. The reflective electrochromic films change between a transparent color and a reflective color depending on whether a voltage is applied or not, and transmit light passing therethrough or reflect it.

Referring to FIG. 3, no voltage is applied to the second electrochromic film 15a on the left hand, and a voltage (V1) is applied to the second electrochromic film 15b on the right hand so that it has been changed to have a reflective color. In addition, a voltage (V2) is applied to the first electrochromic films 16 in order to adjust the intensity of light.

Accordingly, in the course of the light propagation along the waveguide 14, the entire light produced in the light source transmits the second electrochromic film 15a to which no voltage is applied, the intensity of the light is changed passing through the first electrochromic films 16, and the entire light is reflected from the second electrochromic film 15b, so that the light exits via an opening 18 formed in the waveguide 14 to stimulate a neuron. The waveguide 14 may include the opening 18 in the path where the light propagates. Alternatively, the waveguide 14 may be made of a transparent material.

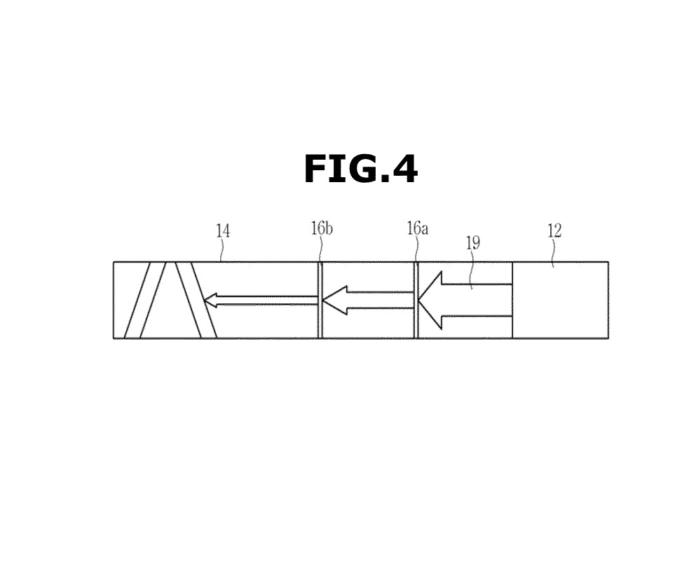

FIG. 4 is a view for illustrating a principle that the optical stimulator according to the exemplary embodiment of the present disclosure adjusts the intensity of light. Referring to FIG. 4, light of an intensity is produced in the light source 12 and propagates along the direction in which the waveguide 14 is extended. The waveguide 14 includes a plurality of electrochromic films therein. First electrochromic films 16a and 16b, whose color is changed depending on the level of voltage applied, may be disposed and spaced apart from each other at a predetermined distance. Each of films is connected to an electrode via a conductive line, such that a voltage level of each of the films may be set.

The first electrochromic film 16 shown in FIG. 3 is inclined at a predetermined angle. However, the first electrochromic film 16 preferably has a face perpendicular to the direction in which the light propagates in order to selectively transmit the light passing therethrough. In the optical stimulator according to the exemplary embodiment of the present disclosure in which the first electrochromic films 16 and the second electrochromic films 15a and 15b are combined, the second electrochromic films 15a and 15b may be disposed to reflect light to a target neuron, and the first electrochromic films 16 may be disposed between the second films 15a and 15b to change the intensity of light.

Accordingly, the first electrochromic film 16 may be either perpendicular or inclined at an angle with respect to the propagation direction of light, depending on the direction in which the second electrochromic films 15a and 15b are designed.

According to the present disclosure, in stimulating a neuron at the same position, it is possible to change the intensity of light transmitted to the neuron by changing the voltage level applied to the first electrochromic films. As a result, it is possible to extract a number of signal recordings from the same neuron according to different stimulation intensities.

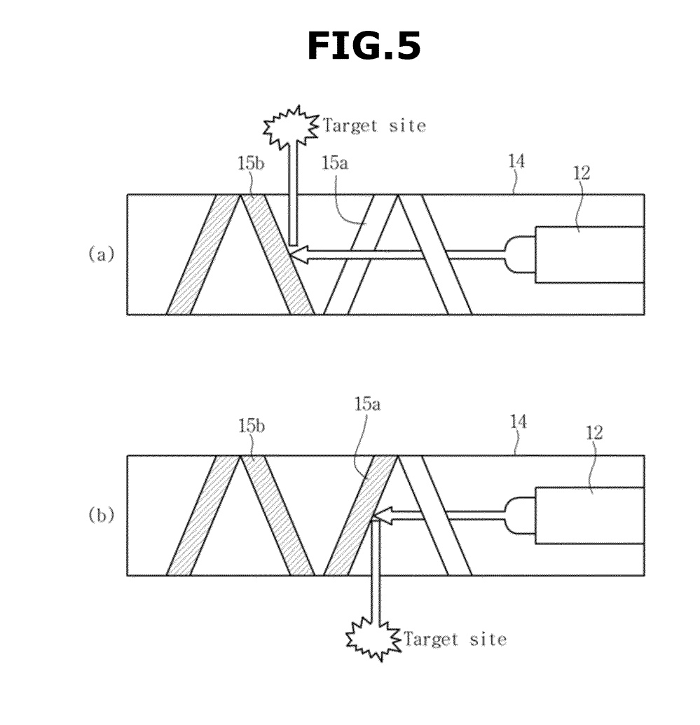

FIG. 5 is a view for illustrating a principle that the optical stimulator according to the exemplary embodiment of the present disclosure changes the path of light. Referring to FIG. 5, light of an intensity is produced in the light source 12 and propagates along the direction in which the waveguide 14 is extended. A plurality of second electrochromic films are disposed in the waveguide 14, each of which is connected to an electrode via a conductive line, such that a voltage is applied to each of the films.

Referring to FIG. 5A, no voltage is applied to the second electrochromic film 15a on the right hand, while a voltage is applied to the second electrochromic film 15b. Referring to FIG. 5B, a voltage is also applied to the second electrochromic film 15a on the right hand. In FIG. 5A, the entire light passes through the second electrochromic film 15a on the right hand, and is reflected from the surface of the second electrochromic film 15b on the left hand, to stimulate a neuron. In FIG. 5B, light is reflected from the second electrochromic film 15a, to stimulate another neuron.

In this manner, it is possible to change the path of the propagating light by applying a voltage to a particular electrochromic film, thereby extracting signal recordings from neurons at multiple sites.

Referring back to FIG. 1, the arrangement of the electrochromic films in the optical stimulator according to the present disclosure may be modified in a variety of ways. As shown in FIG. 1, the first electrochromic films may be disposed and spaced apart from one another by a predetermined distance, and a side face of each of the first electrochromic films may come in contact with a side face of the respective second electrochromic film. In addition, a bottom face or a top face of each of the first electrochromic films may come in contact with a bottom face or a top face of the respective second electrochromic films, forming a predetermined angle. If the side face of the each of the first electrochromic films comes in contact with the side face of the respective second electrochromic films, light propagates along the side face of the waveguide. If the top or bottom face of the each of the first electrochromic films comes in contact with the top or bottom face of the respective second electrochromic films, light propagates along the top or bottom face of the waveguide

Accordingly, as shown in FIG. 1, each of the second electrochromic films disposed between the first electrochromic films comes in contact with the side, top or bottom face of the first electrochromic films, so that the light may propagate along all of the directions of the waveguide.

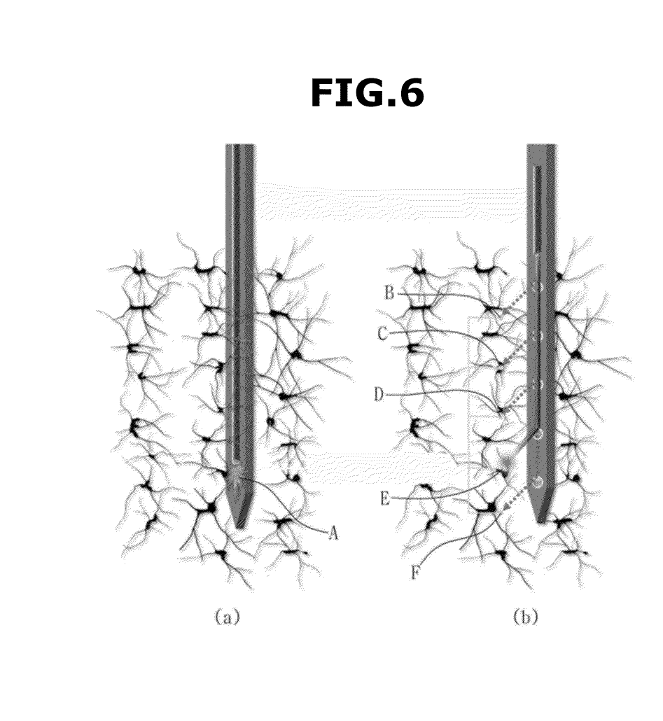

FIGS. 6A and 6B are views for comparing an existing optical stimulator with the optical stimulator according to the exemplary embodiment of the present disclosure. Referring to FIG. 6A, in the existing optical stimulator, light propagates only from the tip of the waveguide and stimulates neurons in area A. In order to stimulate another neurons, the optical stimulator has to be inserted into another position.

In contrast, referring to FIG. 6B, the optical stimulator according to the exemplary embodiment of the present disclosure is capable of stimulating neurons at several positions such as areas B, C, D, E and F from the position where it is inserted.

As described above, the optical stimulator according to the present disclosure utilizes transnnissivity and reflectivity of electrochromic films in the waveguide. Specifically, the optical stimulator according to the present disclosure can stimulate neurons at several depths in different directions by changing the intensity of the propagating light and the path of the light, by adjusting voltage applied to the electrochromic films connected to desired portions.

As an application to optogenetics, the optical stimulator according to the exemplary embodiment of the present disclosure is able to perform measurement and analysis of neural signals more precisely with higher resolution, because it can stimulate neurons at several positions in the depth direction and can measure neural signals at the same depth with different light intensities.

According to the present disclosure, the optical stimulator utilizes transnnissivity and reflectivity of electrochromic films in the waveguide. Specifically, the optical stimulator according to the present disclosure can stimulate neurons at several depths in different directions by changing the intensity of the propagating light and the path of the light, by adjusting voltage applied to the electrochromic films connected to desired portions.

When the optical stimulator according to exemplary embodiment of the present disclosure is applied to optogenetics, it is possible to stimulate neurons at several positions in the depth direction rather than neurons in a single stimulation position, such that a neuronal signal can be measured and analyzed more precisely.

According to the present disclosure, light can propagate to a particular position in the depth direction, and a recording electrode is disposed at a position where the propagation of light is changed, such that a signal generated from a target neuron can be acquired more closely.

Although the exemplary embodiments of the present disclosure have been disclosed for illustrative purposes, those skilled in the art would appreciate that various modifications and substitutions may be made without departing from the scope and spirit of the disclosure. For example, elements of the exemplary embodiment of the present disclosure may be modified. Such modifications and substitutions are also construed as falling within the scope of the present disclosure as defined by the appended claims.

What is claimed is:

1. An optical stimulator inserted into a biological tissue for obtaining a neural signal by stimulating a small group of neuron with a propagating light, the optical stimulator comprising:
a main unit comprising electrodes, and electrical terminals connected to the electrodes;
a waveguide extended from a side of the main unit and comprising electrochromic films; and
a light source disposed in the main unit and producing light along a direction in which the electrochromic films formed in the waveguide are arranged,
wherein each of the electrical terminals is connected to the respective electrochromic films, and the electrochromic films change a propagation path of light reaching the electrochromic films upon a voltage being applied thereto from the electrical terminals, to stimulate a neuron.

2. The optical stimulator of claim 1, wherein the electrochromic films comprises first electrochromic films for changing the intensity of light, and second electrochromic films for reflecting light reaching it, wherein the first and second electrochromic films are arranged in the waveguide.

3. The optical stimulator of claim 2, wherein the first electrochromic films have a face perpendicular to the propagation direction of light.

4. The optical stimulator of claim 2, wherein the second electrochromic films are inclined at a predetermined angle with respect to the propagation direction of light.

5. The optical stimulator of claim 2, wherein a color of the first electrochromic films is changed upon a predetermined voltage being applied thereto from the electrical terminals and selectively absorb light reaching them, and the first electrochromic films change the intensity of the light depending on a change in the voltage level.

6. The optical stimulator of claim 2, wherein the second electrochromic films completely transmit light reaching them upon a voltage of 0 V being applied thereto, and the color of the second electrochromic films is changed upon a predetermined voltage being applied thereto from the electrical terminals, to completely reflect the light reaching them.

7. The optical stimulator of claim 2, wherein the waveguide comprises a recording electrode on a face where light reflected from the second electrochromic films propagates.

8. The optical stimulator of claim 1, wherein the electrochromic films are inclined with respect to a propagating light with a predetermined angle, and changes a path of the propagating light toward a top face, a bottom face or a side face of the waveguide depending on the angle.
